SEQUENCE LISTING (7 sequences)

SEQ ID NO: 1 - DNA, 70 bp, Artificial Sequence
  Tn5 gene 5′ primer
catacacagccatacatacgcgtgtggccgctctgcctctctcttgcaggtatggacagcaagcgaaccg

SEQ ID NO: 2 - DNA, 71 bp, Artificial Sequence
  Tn5 gene 3′ primer
atcagggtgacccctacgttacactcctgtcaccaaggagtgggagggactcagaagaactcgtcaagaag

SEQ ID NO: 3 - DNA, 109 bp, Artificial Sequence
  kanamycin PCR primer
tgtgtgaaattgttatccgctcacaattccacacaacatacgagccgggtgccagggcgtgcccttgggctccccgggcgcgtactggttggtcgacactagtattacc

SEQ ID NO: 4 - DNA, 82 bp, Artificial Sequence
  Kanamycin PCR primer
caatacgcaaaccgcctctccccgcgcgttggccgattcattaatgcagcggcgcgcctaggtggaccagttggtgattttg

SEQ ID NO: 5 - DNA, 40 bp, Artificial Sequence
  40 bp core region
cgggtgccagggcgtgcccttgggctccccgggcgcgtac

SEQ ID NO: 6 - DNA, 20 bp, Artificial Sequence
  chicken DNA primer
agccatacatacgcgtgtgg

SEQ ID NO: 7 - DNA, 20 bp, Artificial Sequence
  chicken DNA primer
ggacatctgagtgggaagtg

(19) United States
(12) Patent Application Publication
(10) Pub. No.: US 2005/0229263 A1
(43) Pub. Date: Oct. 13, 2005

(54) Transgenesis by sperm-mediated gene transfer

(76) Inventor: Roland Buelow, Palo Alto, CA (US)

Correspondence Address: HELLER EHRMAN LLP, 275 MIDDLEFIELD ROAD, MENLO PARK, CA, 94025-3506, US

(21) Appl. No.: 10/966,007

(22) Filed: Oct. 14, 2004

Related U.S. Application Data
(60) Provisional application No. 60/511,620, filed on Oct. 14, 2003.

(51) Int. Cl. A01K 67/00

(52) U.S. Cl. 800/8; 800/21

(57) ABSTRACT

The invention relates to improved methods to integrate heterologous DNA into the genome of non-human animals. The transgenic constructs contain a site recognized by a site specific recombinase which can be used to catalyze the insertion of the transgene into the animal's genome. The transgenic constructs and site specific recombinase are introduced into oocytes using sperm. Fertilized oocytes are maintained under conditions that allow recombinase-mediated integration of the transgene into the genome of the non-human animal and development to term. Transgenic animals with transgenes integrated by a recombinase express transgenes at higher levels compared to transgenic animals with randomly integrated transgenes.

### CROSS-REFERENCE TO RELATED APPLICATIONS

This is a non-provisional application filed under 37 CFR 1.53(b), claiming priority under USC Section 119(e) to provisional Patent Application Ser. No. 60/511,620 filed Oct. 14, 2003.

### BACKGROUND OF THE INVENTION

1. Field of the Invention

The present invention concerns methods and means to produce transgenic animals. The invention specifically relates to improved methods to integrate heterologous DNA into the genome of animals. The transgenic constructs contain a site recognized by a site specific recombinase which can be used to catalyze the insertion of the transgene into the animal's genome. The transgenic constructs and site specific recombinase are introduced into oocytes using sperm. Fertilized oocytes are maintained under conditions that allow recombinase-mediated integration of the transgene into the genome of the non-human animal and development to term. Transgenic animals with transgenes integrated by a recombinase express transgenes at higher levels compared to transgenic animals with randomly integrated transgenes.

2. Description of the Related Art

Using recombinant DNA technology, heterologous DNA sequences can be inserted into the genome of an organism. A variety of different procedures have been described, including sperm mediated gene transfer.

The potential use of sperm cells as vectors for gene transfer was first suggested by Brackett et al., Proc., Natl. Acad. Sci. USA 68:353-357 (1971). This was followed by reports of the production of transgenic mice and pigs after in vitro fertilization of oocytes with sperm that had been incubated by naked DNA (Lavitrano et al., Cell 57:717-723 (1989) and Gandolfi et al. Journal of Reproduction and Fertility Abstract Series 4, 10 (1989)), although other laboratories were not able to repeat these experiments (Brinster et al. Cell 59:239-241 (1989) and Gavora et al., Canadian Journal of Animal Science 71:287-291 (1991)). Since then, there have been several reports of successful sperm mediated gene transfer in chicken (Nakanishi and Iritani, Mol. Reprod. Dev. 36:258-261 (1993)); mice (Maione, Mol. Reprod. Dev. 59:406 (1998)); and pigs (Lavitrano et al. Transplant. Proc. 29:3508-3509 (1997); Lavitrano et al., Proc. Natl. Acad. Sci. USA 99:14230-5 (2002); Lavitrano et al., Mol. Reprod. Dev. 64-284-91 (2003)). Similar techniques are also described in U.S. Pat. No. 6,376,743; issued Apr. 23, 2002; U.S. Patent Publication Nos. 20010044937, published Nov. 22, 2001, and 20020108132, published Aug. 8, 2002.

Unfortunately, to produce transgenic animals, most transformation procedures, including sperm-mediated gene transfer, produce low frequencies of transformed germinal cells (oocytes, spermatoza, zygotes, spermatogonia, etc.) or stem cells that contain the introduced DNA inserted in their genomes. Additionally, the random insertion of introduced DNA into the genome of host cells can be lethal if the foreign DNA happens to insert into, and thus mutate, a unique vital native gene in a critical manner. Furthermore, random integration often results in low expression f transgenes in transgenic animals. Therefore, despite its relative simplicity, more wide-spread use of the sperm-mediated gene transfer technique,

Therefore, for the production of transgenic animals by gene transfer, there is a need for a method that can efficiently integrate heterologous DNA into the genome of a variety of animals, and results in a high frequency of founder animals that express high amount of transgene-encoded product(s).

### SUMMARY OF THE INVENTION

In one aspect, the present invention concerns a method for recombinase-mediated integration of a transgene into the genome of a non-human animal, said method comprising:
(i) incubating a sperm cell with a transgene construct containing a first recombination site under conditions such that said transgene construct is bound to or introduced into the sperm cell,
(ii) fertilizing an oocyte with the sperm cell carrying the transgene construct, wherein the genome of the sperm or the oocyte comprises a second recombination site, in the presence of a recombinase, and
(iii) maintaining the embryo developed from the oocyte under conditions that allow recombination between the two recombination sites, wherein the recombination is mediated by said recombinase, and the result of the recombination is integration of one or several copies of said transgene construct into the genome of embryonic cells.

In a particular embodiment, the foregoing method further comprises the steps of:
(iv) maintaining the embryo under conditions that allow development to term, and
(v) identifying at least one offspring with one or several cells that carry the transgene integrated into its/their genome.

In one embodiment, the recombinase is in the form of DNA comprising an expression cassette encoding and capable of expressing it.

In another embodiment, the recombinase is in the form of mRNA encoding a recombinase polypeptide.

In yet another embodiment, the recombinase is in the form of a polypeptide.

The recombinase can, for example, be a site-specific recombinase expressed by a phage, such as, for example, a ΦC31, TP901-1- or R4 recombinase. Alternatively, the recombinase can, for example, be a site specific recombinase selected from the group consisting of Cre-recombinase, Cre-like recombinase, Flp recombinase, and R recombinase.

In a particular embodiment, the recombinase facilitates recombination between two recombination sites that share more than 90% sequence identity.

In another embodiment, the recombinase facilitates recombination between two recombination sites that share less than 90% sequence identity.

In a different embodiment, the recombinase facilitates recombination between a bacterial genomic recombination site and a phage recombination site, where the bacterial genomic recombination site can, for example, be attB and an exemplary phage recombination site is attP.

In a specific embodiment, the first recombination site comprises an attB site, and the second recombination site comprises a pseudo-attP site.

In another specific embodiment, the first recombination site comprises a pseudo-attB site, and the first recombination site comprises an attP site.

In a further embodiment, the recombinase is encoded by ΦC31 or phage R4 or TP901-1.

In a still further embodiment, the recombinase-mediated recombination results in a site that is no longer a substrate for the recombinase.

The recombinase can, for example, be introduced into sperm and/or oocyte before introduction of the transgene construct, and it is possible to introduce more than one transgene constructs.

In a different embodiment, the recombinase is introduced into sperm and/or oocyte concurrently with introduction of a transgene construct comprising one or more transgenes, but it is also possible to introduce the recombinase into the sperm and/or oocyte after introduction of a transgene construct comprising one or more transgenes.

In a further embodiment, the sperm is incubated with DNA complexed with one or more reagents enhancing cellular binding or uptake of DNA and/or with one or more reagents protecting DNA from degradation. The recombinase can also be modified in a way to facilitate transport into the cell nucleus.

The DNA can be linear or circular.

In order to enhance the introduction of he recombinase into the sperm, the sperm may be permeabilized by electroporation, freezing, mechanical or chemical treatment.

In a further aspect, the invention concerns a sperm cell carrying a transgene construct comprising a transgene and a first recombination site recognized by a site-specific recombinase. In a specific embodiment, the sperm cell further comprises a second recombination site in its genome. In another embodiment, the first and second recombination sites present in the sperm cell are recognized by a recombinase selected from the group consisting of ΦC31, TP901-1, R4, Cre-recombinase, Cre-like recombinase, Flp recombinase, and R recombinase.

In a still further aspect, the invention concerns a non-human oocyte fertilized with a transgene construct comprising a transgene and a first recombination site recognized by a site-specific recombinase. In a specific embodiment, the non-human oocyte further comprises a second recombination site in its genome. In another embodiment, the first and second recombination sites are recognized by a recombinase selected from the group consisting of ΦC31, TP901-1, R4, Cre-recombinase, Cre-like recombinase, Flp recombinase, and R recombinase.

In another aspect, the invention concerns a transgenic non-human animal generated with the method described above.

### BRIEF DESCRIPTION OF THE DRAWINGS

FIG. 1 is a schematic depiction of a construct containing a humanized light chain immunoglobulin locus and an attB site. The chicken light chain locus was modified.

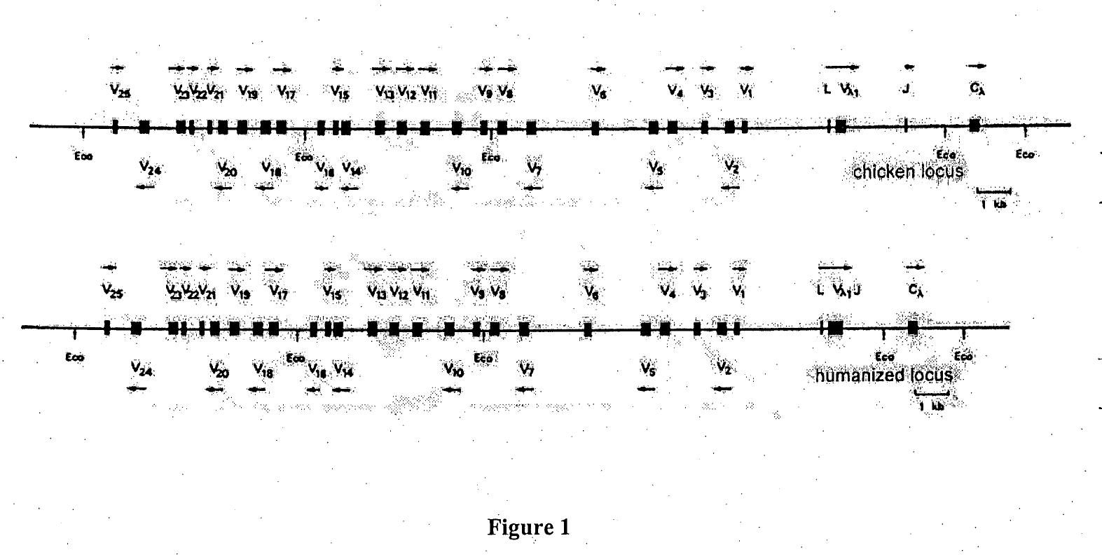

### DETAILED DESCRIPTION

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “sperm” is used to refer to a male gamete cell and includes, without limitation, spermatogonia, primary and secondary permatocytes, spermatids, differentiating spermatids, round spermatids, and spermatozoa.

The term “oocyte” is used to refer to a female gamete cell, and includes primary oocytes, secondary oocytes, and mature, unfertilized ovum.

The term “animal” is used herein in the broadest sense, and includes, but is not limited to, mammals, such as, humans; non-human primates, e.g., apes and monkeys; rodents, e.g., mice, rats, guinea pig, hamster; farm animals, such as cattle, rabbits; swine; sheep; goats; horses; and donkeys; as well as birds, e.g., chickens, turkeys, ducks, geese and the like; and fish.

The term “transgenic (non-human) animal” refers to any (non-human) animal in which one or more cells contain heterologous nucleic acid introduced by human intervention.

The term “transgene construct” is used herein to refer to a double-stranded polynucleotide molecule, which contains a structural gene of interest, a site recognized by a recombinase, and typically other sequences facilitating gene transfer. The transgene construct can, for example, be a vector (plasmid) comprising the gene of interest operatively linked to regulatory sequences and a recombination site (e.g., an attB, attP, pseudo-attB, or pseudo-attP site).

The terms “polynucleotide” and “nucleic acid” are used interchangeably, and, when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “exogenous nucleic acid” or “foreign nucleic acid” or “recombinant nucleic acid” or grammatical equivalents thereof are used to refer to nucleic acid which encodes proteins not ordinarily made in appreciable amounts in the target cells. Thus, exogenous nucleic acid includes nucleic acid which is not ordinarily found in the genome of the target cell. Exogenous nucleic acid also includes nucleic acid which is ordinarily found within the genome of the target cell, but is in a form which allows for the expression of proteins which are not ordinarily expressed in the target cells in appreciable amounts. Alternatively, the exogenous nucleic acid may encode a variant or mutant form of a naturally-occurring protein.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Native antibodies and immunoglobulins” are usually heterotetrameric glycoproteins of about 150,000 daltons, composed of two identical light (L) chains and two identical heavy (H) chains. Each light chain is linked to a heavy chain by covalent disulfide bond(s), while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al., J. Mol. Biol. 186:651 (1985); Novotny and Haber, Proc. Natl. Acad. Sci. U.S.A. 82:4592 (1985)).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, connected by three CDRs. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

The term “monoclonal antibody” is used to refer to an antibody molecule synthesized by a single clone of B cells.

The term “polyclonal antibody” is used to refer to a population of antibody molecules synthesized by a population of B cells.

The terms “a humanized antibody” and “a humanized immunoglobulin”, as used herein, mean an immunoglobulin molecule comprising at least a portion of a human immunoglobulin polypeptide sequence (or a polypeptide sequence encoded by a human immunoglobulin gene segment). The humanized immunoglobulin molecules of the present invention can be isolated from a transgenic non-human animal engineered to produce humanized immunoglobulin molecules. Such humanized immunoglobulin molecules are less immunogenic to primates, especially humans, relative to non-humanized immunoglobulin molecules prepared from the animal or prepared from cells derived from the animal.

The term “Ig gene segment” as used herein refers to segments of DNA encoding various portions of an Ig molecule, which are present in the germline of animals and humans, and which are brought together in B cells to form rearranged Ig genes. Thus, Ig gene segments as used herein include V gene segments, D gene segments, J gene segments and C region gene segments.

The term “human Ig gene segment” as used herein includes both naturally occurring sequences of a human Ig gene segment, degenerate forms of naturally occurring sequences of a human Ig gene segment, as well as synthetic sequences that encode a polypeptide sequence substantially identical to the polypeptide encoded by a naturally occurring sequence of a human Ig gene segment. By “substantially” is meant that the degree of amino acid sequence identity is at least about 85%-95%. In a particular embodiment, the human Ig gene segment renders the immunoglobulin molecule non-immunogenic in humans.

B. Detailed Description

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

Sperm-mediated gene transfer (SMGT) is based on the finding that sperm cells bind and/or internalize exogenous DNA and can, therefore, be used as a vector for transmitting the exogenously introduced DNA to the zygote. The gene of interest can be introduced into the sperm cells as naked DNA by simply incubating the sperm with the DNA, but this method of gene delivery is rather inefficient. Therefore, more commonly, DNA is introduced into the sperm cells by other techniques of gene transfer. More specifically, in the process of the present invention, the transgene is delivered into the sperm cell as part of a transgene construct, which contains a recombination site recognized by a site-specific recombinase. The transgene is typically under the control of a promoter that regulated its expression. In addition to the transgene, promoter, and the recombination site, the transgene construct may contain a 3′ untranslated region downstream of the transgene sequence. The 3′ untranslation region can function to stabilize the RNA transcript of the expression system, and, as a result, increase the yield of the protein encoded by the transgene. The transgene construct may additionally or alternatively include a 5′ untranslated region, which may be taken, for example, from the same control region as the promoter, but may also be different, either natural or synthetic sequence.

The transgene construct can be delivered into the sperm cells by any of the known gene delivery methods, including, without limitation, by permeabilizing the sperm cells, e.g., by electroporation, freezing or chemical or mechanical treatment followed by incubation with transgene construct, transformation with liposomes containing exogenous nucleic acid, biolistic nucleic acid delivery (i.e., loading the nucleic acid onto gold or other metal particles and shooting or injecting into the cells), and the like. For example, gene delivery by electroporation to bovine (Gagne et al., Mol. Reprod. and Develop. 29:6-15 (1991)), chicken (Nakanishi and Iritani, 1993, supra), carp, the American catfish and tilapia (Muller et al., Mol. Mar. Biol. Biotechnol. 1:276-281 (1992)), Zebrafish (Patil and Khoo, J. Exp. Zool. 274:121-129 (1996)), finfish and shellfish (Tsai, H. J., Mol. Reprod. Dev. 56(S2):281-284 (2000)) sperm has been described. The successful use of liposomes for gene transfer into mouse sperm has been reported, for example, by Bachiller et al., Mol. Reprod. Dev. 30:194-200 (1991), and Sasaki et al., Hinyokika Kiyo 46:591-5 (2000).

In an alternative embodiment, the transgene construct is bound to the sperm cell.

The sperm containing or otherwise carrying the transgenic construct is then used to fertilize an oocyte, where the genome of the sperm or of the oocyte comprises a second recombination site. The oocytes may be fertilized by methods known in the art, including, without limitation, by electrofusion or intracytoplasmic sperm injection.

Recombination between the two recombination sites is facilitated by a recombinase. The recombinase may be introduced into germ cells as anon-viral or viral vector encoding the recombinase. Alternatively, the recombinase may be introduced as an enzymatically active protein or through introduction of the encoding messenger RNA (mRNA).

Site-specific recombinases catalyze homologous recombination between two nucleic acid, e.g., DNA segments. These recombinases recognize very specific sequences in both partners of the recombination. While the mechanism of catalysis might be different for different types of site-specific recombinases, they are all included herein, and are suitable for the practice of the present invention. If the recombinase facilitates recombination between two recombinase-specific recognition sites, the recombinase may, for example, be Cre, Flp, or the like. Cre and Flp are the two most commonly used enzymes, which only act on very specific DNA sequences. Cre ctalyzes the recombination of DNA between 34-base pair long loxP sites, while Flp targets the frt site. The use of Cre recombinase for site-specific recombination of DNA in eukaryotic cells is described in U.S. Pat. No. 4,959,317. Site specific recombination in general is described in U.S. Pat. No. 4,673,640. Cre/loxP based cloning systems are commercially available, for example, from BD Biosciences-Clontech, Palo Alto, Calif. (Creator™), oe Invitrogen, Carlsbad, Calif. (Echo™).

In another embodiment, the recombinase may be a site-specific recombinase encoded by a phage selected from the group consisting of ΦC31, TP901-1, and R4. ΦC31 and R4 belong in the integrase family of site-specific recombinases, while TP901-1 belongs to the family of extended resolvases. The R4 integrase is a site-specific, unidirectional recombinase derived from the genome of phage R4 of Streptomyces parvulus. The site-specific integrase TP901-1 is encoded by phage TP901-1 of Lactococcus lactis subsp. cremoris. The recombinase may catalyze recombination between a bacterial genomic recombination site (attB) and a phage genomic recombination site (attP), or the first site may comprises a pseudo-attB site and/or the second site may comprises a pseudo-attP site, or vice-versa.

Recombinase specific recognition sequences can be introduced into an animal's genome or into the germcell's genome using standard transgenic technology including viral and non-viral vectors and other gene transfer techniques discussed above. Preferably, one or several such sites are introduced into the animal's genome in combination with a marker gene that allows analysis of gene expression levels at the integration site. Potential marker genes are luciferase, Green-Fluorescence-Protein (GFP), Chloramphenicol-Acetyl-Transferase, and the like. Subsequent to the generation of animals comprising a recombinase specific recognition sequence at a site that allows high gene expression, germ cells of such animal can be used for the recombinase mediated integration of a construct comprising one or several transgenes.

The embryo may then be maintained under conditions that allow development to term. Transgenic founder animals with one or several cells carrying the transgene can then be identified using various methods for the detection of the transgene.

The transgene can be any gene encoding a desired polypeptide. In a particular embodiment, the transgene encodes a humanized antibody chain, fragment of such chains, linear antibodies, diabodies, and the like. The method of the present invention can also be used to introduce multiple genes into genome of the target animal for coexpression. Thus, for example, genes encoding multiple antibody chains (e.g., heavy and light chains) or antibody fragments can be introduced into transgenic animals by sperm-mediated gene transfer.

In a particular embodiment, the sperm-mediated gene transfer of the present invention is used to introduce a humanized or human immunoglobulin (Ig) heavy chain locus into the recipient animal. In another embodiment, the sperm-mediated gene transfer of the present invention is used to introduce a humanized or human light chain locus into the recipient animal.

Once a transgenic non-human animal capable of producing diversified humanized or human immunoglobulin molecules is made, human(ized) immunoglobulins and human(ized) antibody preparations against an antigen can be readily obtained by immunizing the animal with the antigen. A variety of antigens can be used to immunize a transgenic host animal. Such antigens include, without limitation, microorganisms, e.g., viruses and unicellular organisms (such as bacteria and fungi), alive, attenuated or dead, fragments of the microorganisms, or antigenic molecules isolated from the microorganisms.

Exemplary bacterial antigens for use in immunizing an animal include purified antigens from Staphylococcus aureus such as capsular polysaccharides type 5 and 8, recombinant versions of virulence factors such as alpha-toxin, adhesin binding proteins, collagen binding proteins, and fibronectin binding proteins. Exemplary bacterial antigens also include an attenuated version of S. aureus, Pseudomonas aeruginosa, enterococcus, enterobacter, and Klebsiella pneumoniae, or culture supernatant from these bacteria cells. Other bacterial antigens which can be used in immunization include purified lipopolysaccharide (LPS), capsular antigens, capsular polysaccharides and/or recombinant versions of the outer membrane proteins, fibronectin binding proteins, endotoxin, and exotoxin from Pseudomonas aeruginosa, enterococcus, enterobacter, and Klebsiella pneumoniae.

Exemplary antigens for the generation of antibodies against fungi include attenuated version of fungi or outer membrane proteins thereof, which fungi include, but are not limited to, Candida albicans, Candida parapsilosis, Candida tropicalis, and Cryptococcus neoformans.

Exemplary antigens for use in immunization in order to generate antibodies against viruses include the envelop proteins and attenuated versions of viruses which include, but are not limited to respiratory synctial virus (RSV) (particularly the F-Protein), Hepatitis C virus (HCV), Hepatits B virus (HBV), cytomegalovirus (CMV), EBV, and HSV.

Therapeutic antibodies can be generated for the treatment of cancer by immunizing transgenic animals with isolated tumor cells or tumor cell lines; tumor-associated antigens which include, but are not limited to, Her-2-neu antigen (antibodies against which are useful for the treatment of breast cancer); CD19, CD20, CD22 and CD53 antigens (antibodies against which are useful for the treatment of B cell lymphomas), (3) prostate specific membrane antigen (PMSA) (antibodies against which are useful for the treatment of prostate cancer), and 17-1A molecule (antibodies against which are useful for the treatment of colon cancer).

The antigens can be administered to a transgenic host animal in any convenient manner, with or without an adjuvant, and can be administered in accordance with a predetermined schedule.

After immunization, serum or milk from the immunized transgenic animals can be fractionated for the purification of pharmaceutical grade polyclonal antibodies specific for the antigen. In the case of transgenic birds, antibodies can also be made by fractionating egg yolks. A concentrated, purified immunoglobulin fraction may be obtained by chromatography (affinity, ionic exchange, gel filtration, etc.), selective precipitation with salts such as ammonium sulfate, organic solvents such as ethanol, or polymers such as polyethyleneglycol.

For making a monoclonal antibody, spleen cells are isolated from the immunized transgenic animal and used either in cell fusion with transformed cell lines for the production of hybridomas, or cDNAs encoding antibodies are cloned by standard molecular biology techniques and expressed in transfected cells. The procedures for making monoclonal antibodies are well established in the art. See, e.g., European Patent Application 0 583 980 A1 (“Method For Generating Monoclonal Antibodies From Rabbits”), U.S. Pat. No. 4,977,081 (“Stable Rabbit-Mouse Hybridomas And Secretion Products Thereof”), WO 97/16537 (“Stable Chicken B-cell Line And Method of Use Thereof”), and EP 0 491 057 B1 (“Hybridoma Which Produces Avian Specific Immunoglobulin G”), the disclosures of which are incorporated herein by reference. In vitro production of monoclonal antibodies from cloned cDNA molecules has been described by Andris-Widhopf et al., “Methods for the generation of chicken monoclonal antibody fragments by phage display”, J Immunol Methods 242:159 (2000), and by Burton, D. R., “Phage display”, Immunotechnology 1:87 (1995), the disclosures of which are incorporated herein by reference.

It is emphasized, however, that the present method is not limited to the delivery of any particular gene, or to any particular human or non-human animal host. Thus, for example, human decay accelerating factor (hDAF) has been produced in pigs for xenotransplantation (Lavitrano et al., Proc. Nat. Acad. Sci. USA 99:14230-14235 (2992)), and can be produced more efficiently by the improved method of the present invention. Similarly, the present technique can be used for the delivery of any gene that finds utility in gene therapy, such as, for example, genes associated with muscular dystrophy, heart disease, various neurological conditions or neurodegenerative diseases. Representative examples of genes that are candidates for gene therapy include, without limitation, genes for NGF (e.g., for the treatment of spinal cord injuries or Alzheimer's disease); VEGF (e.g., to stimulate angiogenesis); IGF-1 (e.g., for the treatment of amylotrophic lateral sclerosis—ALS); CF (for the treatment of cystic fibrosis); glucocerebrosidase enzyme (GC) (for the treatment of Gaucher disease); tumor suppressor genes, such as p53 (cancer therapy).

The transgenic animals of the present invention can be knock-outs or knock-ins. In a knock-out, preferably the target gene expression is undetectable or insignificant. Knocking out of a target gene can be achieved by a variety of mechanisms, including, for example, introduction of a disruption of the coding sequence, e.g., by insertion of one or more stop codons, insertion of a DNA fragment, etc., deletion of coding sequence, substitution of stop codons for coding sequence, etc. A “knock-in” of a target gene means an alteration in a host cell genome that results in altered expression or function of a native target gene. Increased (including ectopic) or decreased expression may be achieved by introduction of an additional copy of the target gene, or by operatively inserting a regulatory sequence that provides for enhanced expression of an endogenous copy of the target gene. These changes may be constitutive or conditional, i.e., dependent on the presence of an activator or repressor.

Further details of the invention will be apparent from the following non-limiting examples.

### EXAMPLE 1

A. Construction of a DNA Fragment Containing a Modified Chicken Light Chain Locus Having a Human Clambda2 Gene Segment and a VJ Gene Segment Encoding a Human VL Domain

A genomic BAC library derived from a jungle fowl chicken was screened with radiolabeled probes specific for chicken light chain Clambda and chicken Vpsi25 (the V gene segment at the very 5′ end of the light chain locus). A BAC clone containing the entire lambda light chain locus was identified. The chicken Cl gene on this BAC clone was replaced with the human Cl2 gene by homologous recombination in E. coli using the pET system (Zhang et al., Nat. Biotechnol. 18(12):1314-7, 2000) as follows.

A first DNA fragment containing a kanamycin selection cassette was generated by PCR using primers specific for Tn5 gene. The 5′ primer (5′catacacagccatacatacgcgtgtggccgctctgcctctctcttgcaggTATGGACAGCAAGCGAAC CG3′) (SEQ ID NO: 1) was designed to include 50 bp at the 5′ end (lower case), derived from the 5′ flanking region of the chicken light chain Cl gene. The 3′ primer (5′atcagggtgacccctacgttacactcctgtcaccaaggagtgggagggacTCAGAAGAACTCGTCAA GAAG3′) (SEQ ID NO: 2) was designed to include about 50 bp at the end (lower case), derived from the 3′ flanking region of the chicken light chain Cl gene.

A second DNA fragment was synthesized using overlapping oligonucleotides wherein the DNA fragment contains from 5′ to 3′, a sequence derived from the 5′ flanking region of the chicken light chain Clambda gene, the human Clambda2 gene, and a sequence derived from the 3′ flanking region of the chicken Clambda gene.

E. coli cells containing the chicken light chain BAC clone were transformed with a recombination plasmid expressing the recE and recT functions under an inducible promotor. Cells transformed with the recombination plasmid were then transformed with the first DNA fragment above and selected afterwards in media containing kanamycin. Clones resistant to kanamycin were identified, and the replacement of the chicken Cl segment by the kanamycin selection cassette via homologous recombination was confirmed by restriction enzyme digest.

In the second homologous recombination step, cells positive for the kanamycin selection cassette were transformed with the second DNA fragment above. Transformed cells were screened for the loss of kanamycin resistance as indicative of the replacement of the kanamycin selection cassette by the human Cl2 gene. The exchange was confirmed by restriction enzyme digest and/or sequence analysis.

The BAC clone containing the chicken light chain locus and the inserted human Clambda2 gene segment was further modified by inserting a rearranged VJ DNA fragment. The rearranged VJ DNA fragment encodes a human immunoglobulin variable domain polypeptide described by Kametani et al. (J. Biochem. 93 (2), 421-429, 1983) as IG LAMBDA CHAIN V-I REGION NIG-64 (P01702). The nucleotide sequence of the rearranged VJ fragment was so designed as to maximize the sequence homology at the nucleotide level to the chicken Vlambda1 sequence published by McCormack et al. (Cell 56, 785-791, 1989). This rearranged VJ DNA sequence is more than 80% identical with known chicken light chain V genes. The rearranged VJ DNA fragment was linked to a 5′ flanking sequence and a 3′ flanking sequence. The 5′ flanking sequence was derived from 5′ of chicken Vlambda1, and the 3′ flanking sequence was derived from 3′ of chicken J. The DNA fragment was subsequently inserted into the chicken light chain locus in E. coli using the pET system. The insertion was performed in such a way that the region on the chicken light chain locus from the 5′ end of the chicken Vlambda1 gene segment to the 3′ end of the chicken J region was replaced with the rearranged, synthetic VJ DNA fragment. Again, this insertion wais accomplished by the replacement of the chicken V-J region with a marker gene, followed by the replacement of the marker gene with the rearranged VJ DNA fragment. The modified BAC clone was amplified and purified using standard procedures.

Subsequently, a 35 kb NotI fragment containing the entire humanized light chain locus was excised and cloned into PGEM13Zf(+) vector (=CLC-pGEM). A kanamycin-resistance cassette (neo) was PCR-amplified using primers attB40-neo.up and attB40-neo.do (5′-TGTGTGAAATTGTTATCCGCTCACAATTCCACACAACATACGAGCCGGGTGCCA GGGCGTGCCCTTGGGCTCCCCGGGCGCGTACTGGTTGGTCGACACTAGTATTA CC-3′ (SEQ ID NO: 3) and 3′-CAATACGCAAACCGCCTCTCCCCGCGCGTTGGCCGATTCATTAATGCAGCGGC GCGC CTAGGTGGACCAGTTGGTGATTTTG-5′, respectively) (SEQ ID NO: 4). The primers contain 47 bp and 50 bp homology regions to pGEM13Zf(+), respectively, and the up-primer additionally a 40 bp-core region (CGGGTGCCAGGGCGTGCCCTTGGGCTCCCCGGGCGCGTAC) (SEQ ID NO: 5) of the attB site. The attB-neo-cassette was inserted into CLC-pGEM by ET cloning. The final construct was called CLCattB.

### EXAMPLE 2

Construction of a ΦC31-Integrase Expression Plasmid.

ΦC31 integrase gene was amplified by PCR using plasmid pSET152 (GI: 17974209) as a template. Oligos were designed in such a way that the encoded integrase protein contains a nuclear loclization site at the C-terminus. An IRES sequence was amplified by PCR using plasmid pGL3R (Stoneley et al., Oncogene 16, 423-428, 1998). Integrase and IRES PCR products were combined by PCR amplification using overlapping oligonucleotides. The PCR product was digested with BamHI and XhoI and cloned into pcDNA3.1. The sequence of the final product (pcDNA3.1-IRES-C31-NLS) was confirmed.

### EXAMPLE 3

Sperm Mediated Gene Transfer Using Integrase for the Generation of Transgenic Chicken Expressing Humanized Immunoglobulins

Sperm from roosters is collected and washed in SFM medium (1 liter contains 6 g BSA, 11.35 g glucose, 10 g Na-citrate, 4.7 g EDTA (2H2O), 3.25 g citric acid (H2O) 6.5 g Trizma, pH 7.4). Lipofectin (BRL-Gibco) (15 ug) is mixed with 5 ug DNA (2.5 ug CLCattB+2.5 ug pcDNA3.1-IRES-C31-NLS) and 10×10⁶ sperm cells and incubated for 2 hours at 18-20° C. Subsequently, 12 to 24 week old hens are inseminated artificially.

Newly laid eggs are incubated until hetching and skin biopsies from 2-5 days old chicken are digested with proteinase K. Proteins are removed by phenol/chloroform extraction and DNA is precipitated with ethanol and resuspended in water. CLCattB in chicken genomic DNA is amplified by PCR using the following primers and conditions: P1 (5′AGCCATACATACGCGTGTGG3′) (SEQ ID NO: 6), P2 (5′GGACATCTGAGTGGGAAGTG3′) (SEQ ID NO: 7); denaturation at 95° C. for 2 minutes, then 35 cycles of 95° C., 55° C., 72° C. (30 seconds each), and a final elongation step at 72° C. for 2 minutes. Subsequently, amplification products are analyzed by gel electrophoresis.

Human I-light chain in the serum of 4 week old transgenic founder chicken is detected by ELISA. Elisa plates (Nunc Maxisorp) are incubated with 100 ul/well anti-human lambda light chain antibody (Sigma, L6522) at 10 ug/ml in coating buffer (4.29 g Na2CO3×10H2O; 2.93 g NaHCO3 ad 1 l H2O, pH 9.6) overnight at 4° C. Unbound antibody is removed by washing the plates four times with 200 μl/well wash buffer (0.05% (v/v) Tween 20 in PBS) and remaining binding sites are blocked by incubating plates with 100 μl/well blocking buffer (0.1% (w/v) BSA in PBS) for 1-2 hours at room temperature. Plates are washed 4 times with wash buffer and incubated with 100 μl serum diluted in 0.1% BSA in PBS for 3 hours at room temperature. Unbound antibody is removed by washing the plates four times and bound antibody is detected with 100 μl/well biotinylated anti-human lambda antibody (Sigma, B0900) diluted in 0.1% BSA in PBS. Following incubation for 1 hour at room temperature plates are washed again four times and 100 μl/well of streptavidin-peroxidase complexes (Sigma, S2834), diluted in 0.1% BSA in PBS is added. After an incubation of 30 minutes, plates are washed again four times and bound peroxidase is detected with 100 μl/well ready-to-use TMB solution (Sigma, T4444). The enzymatic reaction is stopped with 100 μl/well 1N H2SO4 and absorbance is measured at 450 nm. Transgenic founder chicken sera contain 1-50 μg/ml human lambda light chain immunoglobulin.

All references cited throughout the disclosure are hereby expressly incorporated by reference.

While the invention has been described with emphasis upon certain specific embodiments, it is be apparent to those skilled in the art that variations and modification in the specific methods and techniques are possible. Accordingly, this invention includes all modifications encompassed within the spirit and scope of the invention as defined by the following claims.

1. A method for recombinase-mediated integration of a transgene into the genome of a non-human animal, said method comprising:
(i) incubating a sperm cell with a transgene construct containing a first recombination site under conditions such that said transgene construct is bound to or introduced into said sperm cell,
(ii) fertilizing an oocyte with the sperm cell carrying the transgene construct, wherein the genome of the sperm or the oocyte comprises a second recombination site, in the presence of a recombinase, and
(iii) maintaining the embryo developed from said oocyte under conditions that allow recombination between the two recombination sites, wherein the recombination is mediated by said recombinase, and the result of the recombination is integration of one or several copies of said transgene construct into the genome of embryonic cells.

2. The method of claim 1, further comprising the steps of
(iv) maintaining the embryo under conditions that allow development to term, and
(v) identifying at least one offspring with one or several cells that carry the transgene integrated into its/their genome.

3. The method of claim 1 wherein said recombinase is in the form of DNA comprising an expression cassette encoding said recombinase.

4. The method of claim 1 wherein said recombinase is in the form of mRNA encoding a recombinase polypeptide.

5. The method of claim 1 wherein said recombinase is in the form of a polypeptide.

6. The method of claim 1, wherein said recombinase is a site-specific recombinase expressed by a phage.

7. The method of claim 6 wherein said recombinase is selected from the group consisting of ΦC31, TP901-1- and R4 recombinases.

8. The method of claim 1, wherein said recombinase is a site specific recombinase selected from the group consisting of Cre-recombinase, Cre-like recombinase, Flp recombinase, and R recombinase.

9. The method of claim 1 wherein said recombinase facilitates recombination between two recombination sites that share more than 90% sequence identity.

10. The method of claim 1 wherein said recombinase facilitates recombination between two recombination sites that share less than 90% sequence identity.

11. The method of claim 1 wherein said recombinase facilitates recombination between a bacterial genomic recombination site and a phage recombination site.

12. The method of claim 11 wherein said bacterial genomic recombination site is attB and said phage recombination site is attP.

13. The method of claim 12, wherein the first recombination site comprises an attB site, and the second recombination site comprises a pseudo-attP site.

14. The method of claim 12, where the first recombination site comprises an pseudo-attB site, and the first recombination site comprises an attP site.

15. The method of claim 13 or 14, wherein the recombinase is encoded by ΦC31 or phage R4 or TP901-1.

16. The method of claim 15, wherein said recombinase-mediated recombination results in a site that is no longer a substrate for the recombinase.

17. The method of claim 1 wherein said recombinase is introduced into sperm and/or oocyte before introduction of said transgene construct.

18. The method of claim 1 wherein more than one transgene construct is introduced.

19. The method of claim 1 wherein said recombinase is introduced into sperm and/or oocyte concurrently with introduction of a transgene construct comprising one or more transgenes.

20. The method of claim 1 wherein said recombinase is introduced into said sperm and/or oocyte after introduction of a transgene construct comprising one or more transgenes.

21. The method of claim 1 wherein said sperm is incubated with DNA complexed with one or more reagents enhancing cellular binding or uptake of DNA.

22. The method of claim 1 wherein said sperm is incubated with DNA complexed with one or more reagents protecting DNA from degradation.

23. The method of claim 1 wherein said recombinase is modified in a way to facilitate transport into the cell nucleus.

24. The method of claim 1 wherein said DNA is linear.

25. The method of claim 1 wherein said DNA is circular.

26. The method of claim 1 wherein said sperm is permeabilized by electroporation, freezing, mechanical or chemical treatment.

27. A sperm cell carrying a transgene construct comprising a transgene and a first recombination site recognized by a site-specific recombinase.

28. The sperm cell of claim 27 further comprising a second recombination site in its genome.

29. The sperm cell of claim 28 wherein said first and second recombination sites are recognized by a recombinase selected from the group consisting of ΦC31, TP901-1, R4, Cre-recombinase, Cre-like recombinase, Flp recombinase, and R recombinase.

30. A non-human oocyte fertilized with a transgene construct comprising a transgene and a first recombination site recognized by a site-specific recombinase.

31. The non-human ococyte of claim 30 further comprising a second recombination site in its genome.

32. The non-human oocyte of claim 31 wherein said first and second recombination sites are recognized by a recombinase selected from the group consisting of ΦC31, TP901-1, R4, Cre-recombinase, Cre-like recombinase, Flp recombinase, and R recombinase.
